SEQUENCE LISTING (1 sequence)

SEQ ID NO: 1 - protein, 11 aa, E. coli
AGNTYWRANCT

(19) United States
(12) Patent Application Publication
(10) Pub. No.: US 2013/0143774 A1
(43) Pub. Date: Jun. 6, 2013

(54) METHODS AND COMPOSITIONS FOR GENERATING POLYNUCLEIC ACID FRAGMENTS

(71) Applicant: The Regents of the University of California, Oakland, CA (US)

(72) Inventors: Paolo Actis, Santa Cruz, CA (US); Muhammad Akram Tariq, Santa Cruz, CA (US); Hyunsung John Kim, Santa Cruz, CA (US); Nader Pourmand, Scotts Valley, CA (US)

(73) Assignee: The Regents of the University of California, Oakland, CA (US)

(21) Appl. No.: 13/693,686

(22) Filed: Dec. 4, 2012

Related U.S. Application Data
(60) Provisional application No. 61/566,946, filed on Dec. 5, 2011.

(51) Int. Cl. C12Q 1/68

(52) U.S. Cl. CPC C12Q 1/6874
USPC 506/27; 536/22.1; 252/182.12

(57) ABSTRACT

Provided are methods and compositions for the generation of broad size distributions of polynucleic acid fragments from larger polynucleic acids. The invention provides unbiased polynucleic acid fragments, i.e. fragments representative of all portions of the larger polynucleic acids. The methods are amendable to automation and are cost-effective. The methods comprise contacting the polynucleic acid sample with a reducing agent and a transition metal in a solvent to form a mixture. An exemplified reducing agent is sodium ascorbate, and an exemplified transition metal is copper.

### CROSS-REFERENCE TO RELATED APPLICATIONS

This application claims priority from U.S. Provisional Patent Application No. 61/566,946 filed on Dec. 5, 2011, which is hereby incorporated by reference.

### STATEMENT OF GOVERNMENTAL SUPPORT

This invention was made with Government support under Contract No. HG000205 awarded by the National Institutes of Health and Contract No. CA143803 awarded by the National Cancer Institute. The Government has certain rights in this invention.

### REFERENCE TO SEQUENCE LISTING, COMPUTER PROGRAM, OR COMPACT DISK

In accordance with “Legal Framework for EFS-Web,” (Apr. 6, 2011) Applicants submit herewith a sequence listing as an ASCII text file. The text file will serve as both the paper copy required by 37 CFR 1.821(c) and the computer readable form (CRF) required by 37 CFR 1.821(e). The sequence listing is hereby incorporated by reference in its entirety. Said ASCII copy, created on Nov. 16, 2012, is named “482.30-1 Seq. Listing.txt” and is 531 bytes in size.

### BACKGROUND OF THE INVENTION

1. Field of the Invention

The present invention relates to the field of polynucleic acid sample preparation and sequencing.

2. Related Art

Presented below is background information on certain aspects of the present invention as they may relate to technical features referred to in the detailed description, but not necessarily described in detail. The discussion below should not be construed as an admission as to the relevance of the information to the claimed invention or the prior art effect of the material described.

Next generation sequencing (NGS) of DNA has revolutionized the field of genomics by providing large volumes of genetic information in a single run. This advancement in sequencing technologies has dramatically accelerated biological and biomedical research and enhanced the understanding of genomes, metagenomes, transcriptomes and interactome¹.

NGS platforms are intended to lower the cost of DNA sequencing of human genomes, with the ultimate goal of elucidating phenotypic variations, comprehending the disease susceptibility and pharmacogenomics, which will facilitate personalized medicine²⁻³.

Concurrent with the development of new sequencing platforms, many groups are now focused on developing robust and cost-effective protocols for generating sequencing libraries. It was recently reported that a semi-automated library preparation was developed where a liquid handling robot was used in conjunction with carboxy terminated magnetic beads that increased the sample preparation production by 8 fold⁴. However, the DNA fragmentation step, which is required by all the currently available NGS platforms, has prevented the full automation of sample preparation to date. Fully automated library preparation will be achieved only when DNA shearing can be added to the work stream of robotic liquid handlers.

Currently, DNA fragmentation for library preparation is achieved by one of four approaches, many of them costly. The earliest methods relied on the physical shearing of genomic DNA; point-sink hydrodynamics that result when a DNA sample is forced through a small hole by a syringe causes random shearing of DNA in the kilobase (kb) fragment size range⁵. This technology cannot efficiently produce smaller sized fragments and is not automatable. A second, commercially available, method is based on DNA shearing induced by nebulization. Fragmentation is achieved by forcing a DNA solution through a small hole in the nebulizer unit. The fragment size can be controlled by altering the speed at which the DNA solution passes through the hole, the pressure of the gas blowing through the nebulizer, the viscosity of the solution, and the temperature⁶. This method generates random DNA fragments, but the application requires high DNA input because of high losses during the nebulization process. To address certain drawbacks in mechanical shearing methods, enzymatic digestion of DNA was considered. dsDNA Fragmentase™ (New England Biolabs, Ipswich, Mass. 01938-2723) generates dsDNA breaks in a time-dependent manner to yield different size fragments. Concerns regarding possible non-random nicking have apparently been addressed, but published data are not yet available. However, this method is known to have sequence specific biases (http(colon slash slash) Epigeneticscommumty.coni12010/06/chip-sequencing-tips-for-small samples/). The fourth and most widely used method exploits acoustic shockwave technology. Adaptive Focused Acoustics (AFA)™ shearing technology is site-independent but it has a broader distribution range for fragments <1 Kb. This proprietary technology is based on shock wave physics, and is said to be based on high frequency, focused ultrasound. It is automatable as a workstation but cannot be integrated into a fully automated library preparation without investment in expensive robotic plate-handlers. Most recently, a transposase-mediated DNA fragmentation was introduced for construction of fragment libraries. This is a rapid method of library preparation, but it introduces significant sequence specific biases⁷.

Size selection is another consideration in library preparation needed to generate DNA of optimal length for sequencing. Current methods for size selection rely on time-consuming agarose gel electrophoresis or commercial systems, such as Caliper's LabChipXT and Sage Science's Pippin Prep. The stand-alone systems have limitations on the amount of starting material and also have limitations on specific size ranges. They also require cartridges that need to be purchased for every 3-4 samples, which can't be easily integrated into an automated library prep pipeline. The final procedures for library preparation involve a series of enzyme reactions for DNA end repair and adaptor ligation that takes the sheared DNA and adds universal sequences at the fragment ends to allow for amplification and hybridization as well as for DNA enrichment. These procedures require a large number of purification steps following each reaction. In addition to the time and labor needed to complete this process, the multiple purification steps result in loss of DNA meaning that larger and larger amounts of starting material are needed.

An average laboratory technician, for example, may take as much as 20 hours to prepare just one sample or up to 4 samples in parallel without increasing the risk of making mistakes. Streamlining this process could dramatically expedite the sequencing pipeline, while reducing outside variability. One means of expediting sample preparation is to enable automated multiplexing and pooling of several small genomes or samples for a single sequencing run. This would allow for studying hundreds of target sequences in hundreds of individuals. Current available sample preparation protocols process only one sample at a time and rely heavily on spin column purification technologies for isolating DNA. This labor-intensive system is not suitable for automation because it requires multiple centrifugation steps. Furthermore, the purification processes as currently performed, can result in significant reductions as well as variability in DNA yield, limiting preparation of samples.

Transition metal cleavage of DNA has been demonstrated in certain contexts. These methods have been typically carried out in buffered solutions. In addition, these methods typically require the use of piperidine to complete the cleavage process.

US 2005/0191682 to Barone et al., published Sep. 1, 2005, entitled “Methods for Fragmenting DNA,” discloses methods for fragmenting and labeling nucleic acids for hybridization. In one embodiment, copper-phenanthroline and a reducing agent are used with sodium ascorbate to fragment single and double stranded DNA. However, this method is used to fragment DNA samples for labeling and hybridization to oligonucleotide arrays. It also produces a plurality of abasic sites in the DNA, which would render the product unsuitable for subsequent sequencing.

Iron (II) EDTA has been used to fragment DNA when adsorbed onto crystalline calcium for the purpose of determining the number of base pairs per helical turn along DNA⁸. Others have used L-Ascorbic acid, by combining with either copper (II) ion or a copper (II)-tripeptide complex to cleave viral DNAs and proteins in order to determine requirements for DNA degradation and protein scission activities⁹. In another study, oxidative double-strand DNA (dsDNA) is reported by prodigiosins in the presence of a redox-active transition metal (Copper) of supercoiled plasmid DNA.¹⁰ Another report suggests that this is likely due to the highly reactive hydroxyl radical reacting with deoxyribose hydrogen atom.” Transition metal cleavage has been used for mapping footprints of DNA protein complexes¹² and for studying the structure of DNA¹³ and RNA¹⁴ in solution.

Consequently, there is a need for compositions and methods for fully automatable random sequence nucleic acid fragmentation.

### BRIEF SUMMARY OF THE INVENTION

In certain aspects, the present invention comprises a method for generating a population of polynucleic acid fragments from a starting polynucleic acid sample wherein sample polynucleic acids have an average length of at least 1200 nt (nucleotides, or base pairs in the case of dsDNA), and wherein the fragments have a first sized fragment population and one or more second sized fragment populations. Yields from input material are sufficient for use in sequencing the input material with sequencing coverage across the whole sample. Fragment populations are not biased by base content or other factors.

The method further comprises the steps of (a) contacting the sample with a reducing agent and a transition metal in a solvent to form a mixture; and, (b) incubating the mixture from step (a) for a predetermined time to cause random fragmentation of sample polynucleic acids substantially along their length, wherein the yield of fragments is at least 0.2% (e.g. 0.2% to 10%) of the sample, and wherein the amount fragments in the first sized fragment population is greater than amounts of fragments in the one or more second sized fragment populations.

In some embodiments of the present invention, the first sized fragment population has is defined by a nominal size that varies by less than about 50 nt. As a non-limiting example, the first sized fragment population may have a nominal size of 150 nt, meaning that the fragments in the defined size population have a size with minima and maxima of about 100 nt and 200 nt, respectively. Nominal sizes (in nt) of, e.g. 100, 150, 200, 250, etc. may be obtained. As further non-limiting examples, the first sized fragment population may have a nominal size of 150 nt, and be present in a greater amount than the one or more second sized populations (e.g. 250-300 nt, 600-800 nt, etc.) in that the first sized population comprises about 4% to about 10% of the fragments, while fragments of other nominal sizes are present in less than two thirds or less than one half that percentage amount.

In some embodiments of the present invention, the transition metal is an ion; in some embodiments of the present invention, the ion is a copper ion or an iron ion. The copper ion is a cupric ion in some embodiments of the present invention.

In some embodiments, the reducing agent is an ascorbic acid salt or derivative; in some embodiments of the present invention, the reducing agent is sodium ascorbate.

In some embodiments, the solvent is an unbuffered aqueous solution. In some embodiments of the present invention, the unbuffered aqueous solution is water.

In some embodiments, the methods do not include a piperidine cleavage step, so the mixture is not contacted with piperidine.

In some embodiments of the present invention, the methods further include isolating a subpopulation of the fragmented polynucleic acids. The subpopulations can include fragment ranges from about 100-200 nt, about 200-300 nt, about 250-300 nt, about 250-350 nt, about 200-400 nt, about 300-400 nt, about 600-800 nt, about 2,000-4,000 nt, or 8,000-10,000 nt. One fragment range can be processed directly from the solution for sequencing library preparation.

In some embodiments, the fragment size of the first fragment size population comprises a lower size in nt of about 200, about 300, about 400, about 500, about 600, about 700, about 800, about 900 or about 1,0000. In some embodiments, the first fragment population comprises an upper size in nt of about 1,000, about 2,000, about 3,000, about 5,000, or about 10,000.

Also provided herein are kits for generating a population of polynucleic acid fragments in a sample having (a) a transition metal and reducing agent in an unbuffered aqueous solution. In an embodiment, the kit includes instructions for use.

Provided herein are methods for preparing a DNA sequencing library, by (a) generating a population of polynucleic acid fragments in a sample according to any of the methods above for generating a population of polynucleic acid fragments in a sample to generate a mixture; (b) contacting the mixture with a solid support, and allowing the population of polynucleic acid fragments from step (b) to bind to the solid support (c) removing unbound polynucleic acid fragments; and (d) releasing the bound polynucleic acid fragments. In some embodiments, the method further includes end polishing the released polynucleic acid fragments. In some embodiments, the solid support is a bead. In some embodiments, the steps are automated.

Provided herein are methods for generating a population of polynucleic acid fragments in a sample by (a) contacting polynucleic acids in the sample with a reducing agent and a transition metal in an unbuffered aqueous solution to form a mixture and (b) incubating the mixture under conditions that fragment the polynucleic acids into a distribution of fragment sizes.

In some embodiments of the present invention, the unbuffered aqueous solution is in water. In some embodiments, the transition metal is an ion, and the ion can be copper ion or an iron ion. In some embodiments, the copper ion is a cupric ion.

Also provided herein are methods for generating a population of polynucleic acid fragments in a sample by (a) contacting polynucleic acids in the sample, wherein the polynucleic acids are an average length of at least 1200 nt, with a reducing agent and a transition metal in a solvent to form a mixture and (b) incubating the mixture under conditions that fragment the polynucleic acids into a distribution of fragment sizes, wherein the method does not include a piperidine cleavage step.

Also provided herein are compositions of a polynucleic acid, a transition metal and reducing agent in an unbuffered aqueous solution.

### BRIEF DESCRIPTION OF THE DRAWINGS

FIG. 1A is a photograph of an electrophoretic gel showing fragmentation of genomic DNA in the range of 100-200 bp. Lane 1, GeneRuler™ 50 by DNA Ladder (Fermentas Inc, Glen Burnie, Md.); lane 2, human genomic DNA (no fragmentation); lane 3, human DNA digested in equimolar (4 M) concentration of CuSO₄ and sodium ascorbate prior to library preparation for Illumina platform; lane 4, human DNA digested in equimolar (6 mM) concentration of CuSO₄ and sodium ascorbate prior to library preparation for SOLiD platform; lane 5. GeneRuler™ 50 by DNA Ladder (Fermentas Inc, Glen Burnie, Md.).

FIG. 1B is a photograph of an electrophoretic gel separating the products of the incubation of a human genomic DNA sample with CuSO₄ and sodium ascorbate. Lane 1, 1 kb DNA Ladder (New England Biolabs NEB); lane 2, human genomic DNA (no fragmentation); lane 3, human DNA digested in equimolar concentration of CuSO₄ and sodium ascorbate (0.5 mM for 10 minutes) for mate-pair library preparation (10 kb fragments); lane 4, human DNA digested in equimolar concentration of CuSO₄ and sodium ascorbate (0.75 mM for 30 minutes) for mate-pair library preparation (5 kb fragments); Lane 5, human DNA digested in equimolar concentration of CuSO₄ and sodium ascorbate (1.5 mM for 10 minutes) for mate-pair library preparation (2 kb fragments); lane 6, 1 kb DNA Ladder (New England Biolabs NEB).

FIGS. 2A and 2B shows a pair of graphs comparing coverage depth (50 kb coverage window) for the E. coli genome from sequencing data of both Illumina (FIG. 2A) and SOLiD (FIG. 2B) platforms for libraries prepared from products of the two different fragmentation methods.

Average Coverage Depth across E. coli genome with 10 kb windows. Fragmentation methods are compared using (a) Illumina data and (b) Solid sequencing data. Both AFA and metal-based fragmentation show similar coverage profiles across the genome. Coverage of randomly generated reads is represented by the (•••) line. All fragmentation methods and sequencing platforms deviate from truly random uniform fragmentation, but SOLiD sequencing samples are more highly uniformly random. It can be seen that the copper method is essentially equivalent to the sonication method in this respect.

FIG. 3 is a graph showing GC content in the 20 bp region flanking fragmentation start site. The dashed line represents the average GC content of the organism. Human samples show large variation among samples.

FIG. 4 is a representation of a bioanalyzer trace of fragmentation products of 0.5 ug of E. coli genomic DNA incubated with equimolar concentrations of CuSO₄ and sodium ascorbate (1.4 mM) at room temperature for 5 minutes.

FIG. 5 is a representation of a bioanalyzer trace of fragmentation products of 1.0 ug of E. coli genomic DNA incubated with 1.4 mM CuSO4 and 1.7 mM sodium ascorbate at room temperature for 5 minutes.

FIG. 6 is a representation of a bioanalyzer trace of fragmentation products of 3.0 ug of E. coli genomic DNA incubated with 1.4 mM CuSO4 and 2.0 mM of sodium ascorbate at room temperature for 5 minutes.

FIG. 7 is a representation of a bioanalyzer trace of fragmentation products of 0.5 ug of Human genomic DNA 0.5 ug of Human Genomic DNA incubated with 1.4 mM CuSO4 and 2.0 mM of sodium ascorbate at room temperature for 5 minutes.

FIG. 8 is a representation of a bioanalyzer trace of fragmentation products of 1.0 ug of Human genomic DNA incubated with 1.4 mM CuSO4 and 2.5 mM of sodium ascorbate at room temperature for 5 minutes.

FIG. 9 is a representation of a bioanalyzer trace of fragmentation products of 3.0 ug of Human genomic DNA incubated with 1.4 mM CuSO4 and 2.8 mM of sodium ascorbate at room temperature for 5 minutes.

### DETAILED DESCRIPTION OF THE INVENTION

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

Ranges: For conciseness, any range set forth is intended to include any sub-range within the stated range, unless otherwise stated. A subrange is to be included within a range even though no sub-range is explicitly stated in connection with the range. As a nonlimiting example, a range of 120 to 250 includes a range of 120-121, 120-130, 200-225, 121-250 etc. The term “about” has its ordinary meaning of approximately and may be determined in context by experimental variability. In case of doubt, “about” means plus or minus 5% of a stated numerical value.

The term “transition metal” means one of the 38 elements in groups 3 through 12 of the periodic table. Transition metals include “d block elements,” Sc, Ti, V, Cr, Mn, Fe, Co, Ni, Cu and Zn. One of the key features of transition metal chemistry is the wide range of oxidation states (oxidation numbers) that the metals can show. For example, iron has two common oxidation states (+2 and +3) in, for example, Fe2+ and Fe3+. Copper can exist as Cu1+ and Cu2+. A transition metal ion in a “lowered oxidation state” refers to an oxidation state lower than the highest common oxidation state, e.g. Cu+1. Transition metals can exist in ionic form as salts.

The term “reducing agent” means a reagent in a reaction removes oxygen, contributes hydrogen, or contributes electrons. Since oxidation and reduction are symmetric processes, always occurring together, there is always an oxidizing agent and a reducing agent in the reaction. In the present disclosure, a reducing agent acts as such in the presence of a transition metal salt.

The term “ascorbic acid reducing agent” means ascorbic acid and the analogues, isomers and derivatives thereof. Such compounds include, but are not limited to, D- or L-ascorbic acid, sugar-type derivatives thereof (such as sorboascorbic acid, y-lactoascorbic acid, 6-desoxy-L-ascorbic acid, L-rhamnoascorbic acid, imino-6-desoxy-L-ascorbic acid, glucoascorbic acid, fucoascorbic acid; glucoheptoascorbic acid, maltoascorbic acid, L-arabosascorbic acid), sodium ascorbate, potassium ascorbate, isoascorbic acid (or L-erythroascorbic acid), and: salts thereof (such as alkali metal, ammonium, or others known in the art), endiol type ascorbic acid, an enaminol type ascorbic acid, a thioenol type ascorbic acid, and an enamin-thiol type ascorbic acid, as described in U.S. Pat. No. 5,089,819 (Knapp), U.S. Pat. No. 2,688,549 (James et al.), U.S. Pat. No. 5,278,035 (Knapp), U.S. Pat. No. 5,384,232 (Bishop et al.), U.S. Pat. No. 5,376,510 (Parker et al.), and U.S. Pat. No. 5,49851 1 (Yamashita et al.).

The term “chelating agent” is used in its customary sense to refer to molecules can form several bonds to a single metal ion. The chelating agent sequesters the metal ion and prevents its further binding.

Exemplary chelating agents include chelating agents are chosen from ethylene-diaminetetraacetic acid (EDTA), nitrilotriacetic acid and ethylenegylcol-bis(.beta.-amino-ethyl ether)-N,N-tetraacetic acid. A chelating agent that has two coordinating atoms is called bidentate; one that has three, tridentate; and so on. EDTA, or ethylenediaminetetraacetate, (—O2CH2)2NCH2CH2N(CH2CO2-)2, is a common hexadentate chelating agent.

The term “automated liquid handing apparatus” is used in its conventional sense to refer to a liquid handing robot capable of pipetting liquids to and from different containers. Such devices are commercially available from companies such as Mettler, IDEX, Thermoscientific, etc.

They typically include computers for pre-programming movement from one tube or well to another, and for controlling input to and discharge from the pipette. Microfluidic devices are also available that can act as an automated liquid handling apparatus.

The term “solid support” or “solid substrate” means a solid material having a surface for attachment of molecules, compounds, cells, or other entities. The surface of a solid support can be flat or not flat. A solid support can be porous or non-porous. A solid support can be a chip or array that comprises a surface, and that may comprise glass, silicon, nylon, polymers, plastics, ceramics, or metals. A solid support can also be a membrane, such as a nylon, nitrocellulose, or polymeric membrane, or a plate or dish and can be comprised of glass, ceramics, metals, or plastics, such as, for example, polystyrene, polypropylene, polycarbonate, or polyallomer. A solid support can also be a bead, resin or particle of any shape. Such particles or beads can be comprised of any suitable material, such as glass or ceramics, and/or one or more polymers, such as, for example, nylon, polytetrafluoroethylene, TEFLON™, polystyrene, polyacrylamide, sepaharose, agarose, cellulose, cellulose derivatives, or dextran, and/or can comprise metals, particularly paramagnetic metals, such as iron. Solid supports may be flexible, for example, a polyethylene terephthalate (PET) film.

The term “end polishing” means, as is understood in the art of sequencing subjecting duplex nucleic acid molecules having staggered single-strand ends to a process by which the ends are made blunted. This can be done enzymatically, such as by using T4 polynucleotide kinase in the presence of the complementary nucleoside triphosphates.

The term “unbuffered” is used in its conventional sense to refer to a solution containing active ingredients and optional nonbuffering salts such as potassium or sodium hydroxide. Chelating agents such as ethylenediaminetetraacetic acid [EDTA] may be optionally included. However, salts having significant buffering capacity are not added to these solutions. These salts are known in the life sciences filed and are exemplified in commercial catalogs, such as Sigma-Aldrich list of buffer/buffer salts at http (colon slash slash) www (dot).sigmaaldrich.com/programs/research-essentials-products.html?TablePage=103942709. These solutions are suitable to suspend polynucleic acids such as genomic DNA, as described below.

### Overview

The present invention concerns methods to generate polynucleic acid fragments from biological samples. (The polynucleic acids used are referred to for convenience as DNA, but can include mRNA and other polynucleic acids. These may be either or both single stranded and double stranded polynucleic acids.) The biological samples may be, e.g., whole chromosomes that are from 51 million by to 245 million by in length. They may be randomly fragmented chromosomes, such as obtained from forensic samples.

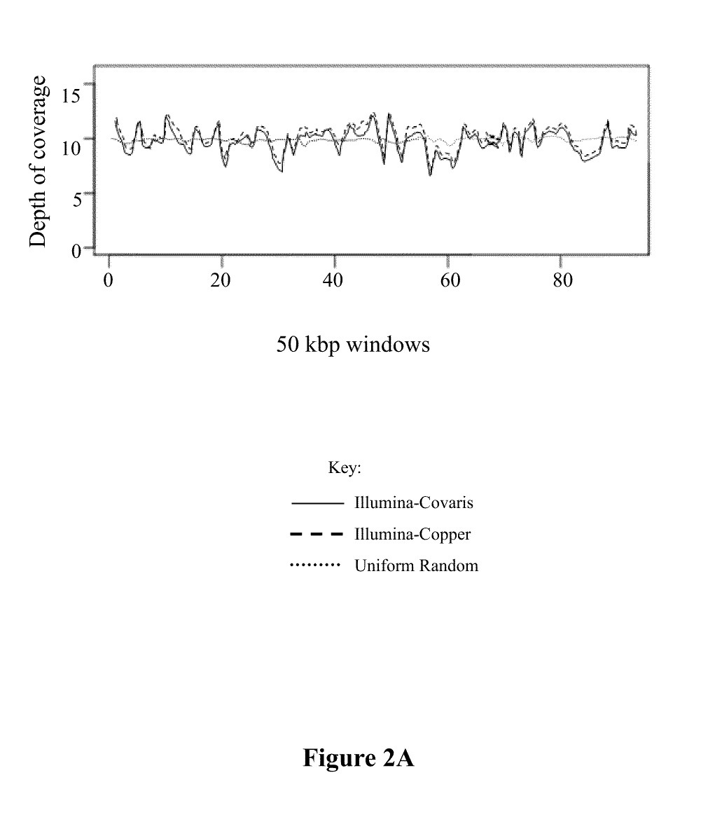

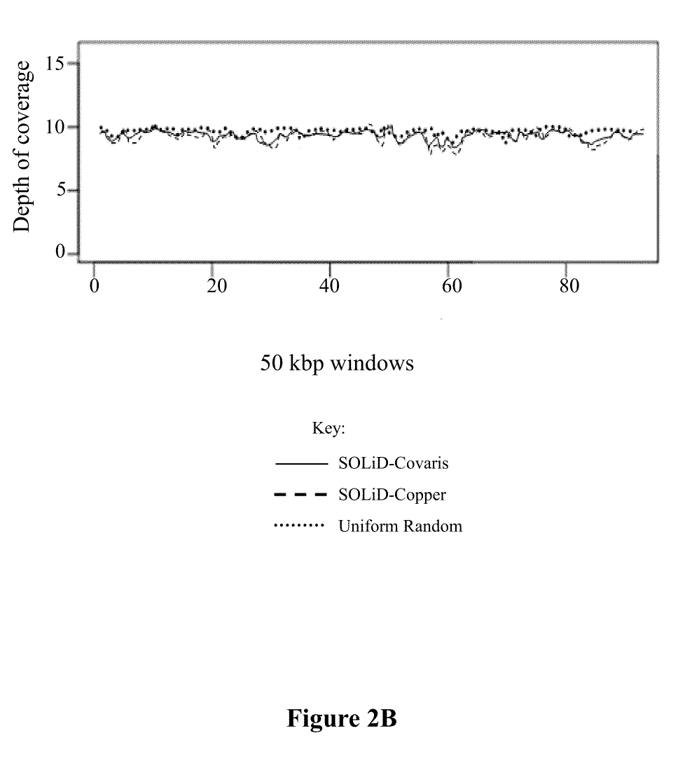

The present methods involve a solvent mixture incubation step for a predetermined time to cause random fragmentation of sample polynucleic acids substantially along their length. The random fragmentation that occurs substantially entirely along the length of the polynucleic acid means that fragments from most if not all regions along the polynucleic acid (e.g. chromosome) are represented, as described for example in connection with FIGS. 2A and 2B. Fragments in a range of sizes (from 100 to 10,000 bp) that are desirable for preparing fragment as well as mate-pair libraries for next-generation sequencing. The size range of a given fragment size population is selected to be within a tolerance of a desired nominal value. For example, if the nominal desired value is 250 nt, the size range may be 200-300 nt. The desired nominal value will be determined by the use made of the fragments, e.g. the manufacturer's recommendations for the particular sequencing library that will be prepared from the fragments. The instant methods generate random fragments of polynucleic acids that are substantially undamaged and are representative of the starting sample so as to provide relatively uniform sequencing results across an assembled sequence. That is, the random fragments comprise a population of fragments from essentially all portions of the sequences of the starting population of polynucleotides and of all sizes. As a result, substantially all portions of starting population that are to be used in sequencing are represented, without a bias for one sequence region to be overly included or omitted. The present methods are highly adaptable to next generation sequencing methods and are automatable, in that they can be carried out entirely in a fluid phase.

The present methods utilize a mixture of a transition metal and a reducing agent, combined with a polynucleic acid to be fragmented in a solvent. In an embodiment, copper in the presence of sodium ascorbate is used. An unexpected result is that the fragmentation reaction performed in an unbuffered aqueous solvent such as water, in contrast to a buffered solution as provided in the prior art, results in a suitable distribution of differently sized fragmentation products. In addition, an unexpected result is that fragmentation without subsequent piperidine cleavage, in contrast to the typical procedure provided in the prior art which includes piperidine cleavage, results in a suitable distribution of differently sized fragmentation products. Provided herein are methods to fragment polynucleic acids to any desired distribution of lengths, such as can be used for fragment libraries as well as mate-pair libraries on NGS platforms. The instant method is readily automatable. Unlike other fragmentation technologies, such as the AFA acoustic shockwave technology, which can only be automated in a serial fashion, the provided methods can be implemented in a highly parallelized manner. Furthermore, the low cost of reagents and their ease of access reduces the cost of the provided methods relative to the prior art alternative methods of fragmenting polynucleic acids.

In one embodiment, the present method optimizes metal-induced oxidative DNA breakage. In some embodiments, cupric ions in presence of sodium ascorbate are used for oxidative DNA breakage for the construction of genomic libraries and ultimately, for use in high-throughput DNA sequencing platforms. Examples of the inventive methods are demonstrated with three different genomic libraries (E. coli, Human and Mouse). The methods are genome independent and do not result in reduced sequence bias. Furthermore, minimal base damage is caused by this fragmentation method as compared with conventional DNA shearing technology.

Genome sequencing centers or laboratories are currently obligated to use two different types of instrument to shear the DNA for fragment libraries and for mate-pair libraries (e.g., a Covaris AFA instrument for fragment libraries, and a Digilab Hydroshear for mate-pair libraries). There is no single method or instrument available that can effectively perform both tasks.

“Mate pair” libraries are, as is known in the art, those used for paired end sequencing. The DNA to be sequenced is sequenced from both ends of a single sequence. That is, a mate-paired library consists of a pair of DNA fragments that are “mates” because they originated from the two ends of the same genomic DNA fragment. For example, in the SOLiD system, the sheared end-repaired template is methylated and capped with EcoP15I CAP adapters. EcoP151 CAP adapters connect the DNA mate-pairs together through a biotinylated internal adapter resulting in DNA circularization. The present method is demonstrated to result in fragments that are not damaged at either end, permitting such use. Further, mate pair libraries can utilize longer fragments of DNA, and the present methods can be fine-tuned according to the average length of fragment desired.

In addition, the present method results in an absence of sequence or base specificity in copper-based DNA fragmentation, unlike conventional AFA technology. Randomness of this DNA fragmentation is demonstrated further by the similar diversity of unique start sites and uniform coverage of all three genomes (E. coli, human and mouse) among libraries prepared using metal-based DNA fragmentation and conventional DNA shearing technology. Furthermore, the present method is genome independent as it can be applied with equal success to E. coli, human, and mouse genomes. Furthermore, in one embodiment, the present method can efficiently be used for construction of both fragments as well as mate-pair libraries.

Fragment size ranges may be expressed as sized “FROM A to B”. In some embodiments populations of fragments of length A to B can be about 100-200 nt, about 200-300 nt, about 250-300 nt, about 250-350, about 200-400 nt, about 300-400 nt, about 600-800 nt, about 2,000-4,000 nt, or 8,000-10,000 nt.

### Fragment Size Distribution

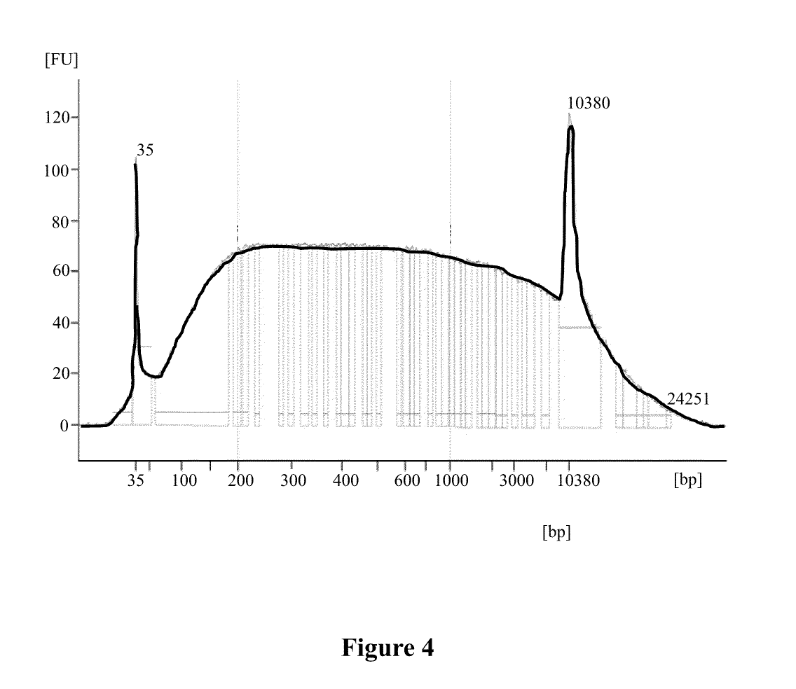

The polynucleic acids from a biological sample (e.g. genomic DNA or genomic RNA from a virus) may be reproducibly fragmented into a predetermined size range, as is shown by the examples below. In particular, Examples 3-8 and accompanying figures show Bioanalyzer trace results of polynucleic acids treated by exemplary methods. As is known in the art, the Bioanalyzer [here, model #2100] (Agilent Technologies)] shows the size and quantity of polynucleic acids (DNA in the examples) in a sample. The trace is essentially an electropherogram that plots size in by of various sizes (or time, which is related to size) against FU, or fluorescence units, where the amount of FU peak represents a quantity of a given size DNA fragment. Differently sized fragments can be separated with high resolution in the device. DNA size markers are provided for use with the device, e.g. at 35 and 1030 by (see FIG. 4).

The controllable nature of the present method can be seen by comparing the results illustrated in FIGS. 4-9. Thus it can be seen e.g. from Example 4 and FIG. 4 that the method produced essentially equal yields of fragments in size ranges between about 9,000 nt. That is, there was approximately the same number of 200 nt fragments as 300 nt fragments, 400 nt fragments, etc. Size ranges are used for convenience; in principle, any size range can be selected in the present method, although for practical purposes a particular size or size range will be selected depending on the use to be made of the fragment population. As also illustrated in the Examples, one can increase the concentration of reducing agent used based on the amount of input polynucleotide sample. That is, the transition metal is used in excess and the reducing agent is provided in an amount based on the amount of input sample.

Notably, as shown in FIGS. 2A and 2B, the fragments are representative of the entire input sample. One may obtain a 10 fold depth of coverage (if desired) for the entire input sample. “Depth of coverage” refers to how may sequence reads are obtained for a given base, and is explained further in e.g. Wang et al. “Scientific Reports 1(55) doi:10.1038/srep00055, “Next generation sequencing has lower sequence coverage and poorer SNP-detection capability in the regulatory regions.” The present methods may be combined with next generation sequencing methods to overcome problems of coverage of areas identified in this paper, viz. CpG islands.

### Fragment Recovery

The present methods further include a step of isolating a subpopulation of the fragmented polynucleic acids. Recovery of selected size fragments can be achieved by methods well known to those skilled in the art. For example, sizing methods such as gel electrophoresis or chromatography can be used. Fragment recovery can take place from solution and fragments can be transferred directly to a reaction mixture used for end polishing and adapter ligation.

### Transition Metals and Salts Thereof

The present methods can use any transition metal. In one aspect in an ionic form (i.e. as a salt). In an aspect, the transition metal is in a composition where the transition metal is in a lowered oxidation state. In an aspect the transition metal is a Cu2+ salt. Other suitable transition metals include Co, Mn or Ni. A variety of transition metal salts can be prepared. For example, one could employ metal alkoxides or acetate or nitrate salts, metal oxides, metal hydroxides, metal halides metal acetylacetonates, metal carbonates, metal carboxylates or metal oxalates. Metals in the form of metallic nanoparticles may also be used. For example, copper nanoparticles as described in U.S. Pat. No. 7,422,620 may be used.

### Solvents

The solvent or combination of solvents used in the present solution can include any solvent or combination of solvents capable of dissolving the transition metal salts and the reducing agent. Such solvents include, for example, alcohols, including, but not limited to methanol, ethanol, isopropanol and butanol. The solvent can be an aqueous solvent and the aqueous solvent can be unbuffered water. In some embodiments, the solvent can include additives that reduce DNA damage, such as salts, nuclease inhibitors, etc. In some embodiments, fragmentation is performed in the absence of piperidine.

### Reducing Agents

The reducing agent(s) can be any molecule that can reduce the oxidative state of a transition metal. Suitable reducing agents include derivatives of ascorbic acid. Reducing agents include, but are not limited to, a hydroquinone, catechol, aminophenol, 3-pyrazolidone such as 1-phenyl-3-pyrazolidone, 1-, d- or isoascorbic acid, ascorbic acid salts, reductone or a phenylenediamine.

### EXAMPLE 1

### Materials and Methods

### DNA Samples

Human blood samples were purchased from Stanford University Blood Bank (Palo Alto, Calif.). Genomic DNA was extracted using QIAamp DNA Blood Maxi Kit (QIAGEN, Valencia Calif., 91355). Mouse pure genomic DNA and E. coli genomic DNA were purchased from Promega Corporation (Madison, Wis.) and USB Corporation (Santa Clara, Calif.) respectively.

### Optimization of DNA Fragmentation Using Metal Ions in Presence of Reducing Agent

Incubation Time and Concentration of Cupric and Ferric Ions and Sodium Ascorbate. 100 mM CuSO₄, FeCl₃, and sodium ascorbate (Sigma-Aldrich, Saint-Louis, Mo.) solutions were prepared using milliQ water. Equimolar concentrations of sodium ascorbate and either CuSO₄ and or FeCl₃ were mixed at varying concentrations ranging from 1 mM to 10 mM, and incubated with 5 ug DNA at room temperature for 30 minutes to induce DNA fragmentation. DNA fragments were recovered as described below. The degree of DNA fragmentation was assessed during gel electrophoresis and was further evaluated by NGS of the library prepared from the fragments (see below).

Effect of Temperature on DNA Fragmentation. 5 ug DNA was incubated in an equimolar solution of 4 mM CuSO₄ and 4 mM Sodium Ascorbate at 25° C., 37° C., 45° C. and 60° C. for 30 minutes to study the effect of temperature on the fragmentation efficiency.

Effect of Oxygen on DNA Fragmentation. 5 ug DNA was mixed with an equimolar solution of 4 mM CuSO₄ solution and 4 mM Sodium Ascorbate and incubated with continuous degassing for 5, 10, 15, 30, 45 and 60 minutes to study the effect of oxygen on the DNA fragmentation.

Effect of DNA Concentration on Fragmentation. DNA of different concentrations (0.5, 1.0, 5.25 and 50 ug) was incubated in an equimolar solution of 4 mM CuSO₄ solution and 4 mM Sodium Ascorbate for 30 minutes at room temperature to assess the efficiency of fragmentation with respect to amount of starting material.

Optimization of DNA Fragment Size for Mate-Pair Libraries. 5 pg of human genomic DNA was incubated at 1.5 mM for 10 minutes to get 2 kb DNA, 0.75 mM for 30 minutes to get 5 kb DNA, and 0.5 mM for 10 minutes to get 10 kb DNA fragments.

Recovery of Fragmented DNA. Fragmented DNA was recovered using three different methods: Charge-switch (Invitrogen Corporation; Carlsbad, Calif.), Carboxy magnetic beads (NorDiag Inc; Westchester, Pa.), and traditional agarose gel as described below. Recovered DNA was quantified by Bioanalyzer [model #2100] (Agilent Technologies; Santa Clara, Calif.) per manufacturer's instructions.

Charge-Switch. Fragmented DNA was purified using charge-switch magnetic beads from a Charge Switch PCR Clean-Up Kit according to manufacturer's instructions (Invitrogen, Catalog #CS12000, Carlsbad, Calif., USA). Briefly, equal volume of purification buffer (N5; pH 5.0, 10 mM NaCl, 0.1% Tween 20) and fragmented DNA sample were mixed (by pipetting) with 10 ul of Charge Switch Magnetic Beads. The mixture was incubated at room temperature for 1 minute. A magnet was applied, and the supernatant was discarded. 150 ul of Washing Buffer (W12) was added to the beads, and mixed by pipetting. A magnet was applied, and supernatant was discarded. The washing was repeated two times. The purified fragmented DNA was eluted using 20 ul of Elution Buffer (E5, 10 mM Tris-HCl, pH 8.5).

Carboxy-Magnetic Beads. The reaction of CuSO₄ and sodium ascorbate was stopped by adding 40 mM EDTA (10 times in concentration to CuSO₄) and then samples were purified using carboxy magnetic beads according to manufacturer's recommendations (NorDiag, Oslo, Norway).

Agarose Gel. Fragmented DNA was run on either 0.8% or 2.0% agarose gel depending upon the required size fragments quickly after treatment with CuSO₄ and sodium ascorbate and fragmented DNA was size selected for next step of library preparation in which DNA is purified by electrophoretic mobility.

Shearing of DNA from Three Different Genomes with Sonication. 2 ug of genomic DNA of each organism (E. coli, Human and Mouse) was loaded into a 6 mm×32 mm Covaris tube. The DNA was sheared to the desired fragment size for library preparation to run on SOLiD and Illumina platforms using a COVARIS (S2) instrument according to manufacturer's recommendation.

### Library Preparation and Sequencing

Approximately 2 ug of fragmented DNA was used for library preparation of each of six samples, comprising the three species (E. coli, mouse, human) fragmented using the metal based system described here, or the Covaris method. Because the three isolation methods proved equivalent, only the Charge Switch samples were used. The fragmented DNA was electrophoresed on 2.5% agarose gel and size selected in the range of 100-200 bp for SOLiD and 250-350 bp for Illumina platforms. The DNA fragments were then blunt-ended through an end-repair reaction and ligated to platform-specific, double-stranded bar-coded adapters using library preparation kits from New England Biolabs (Ipswich, Mass. 01938-2723). All the libraries were prepared through a semi-automated procedure using NorDiag Magnatrix 8000 plus liquid-handling Robot (NorDiag, Oslo, Norway). The end-repair, dA-tailing (for Illumina-based libraries only), ligation of platform-specific adaptors and purification reactions required for library preparation steps were done in an automated fashion, while gel purification and library amplification were done manually.

The six different bar-coded genomic DNA libraries (E. coli, Human and Mouse libraries prepared by Covaris and prepared by the disclosed metal-based fragmentation system) were pooled together in equal concentration to avoid any variation and subjected to sequencing in the same quad of one slide on a SOLiD sequencer (Applied Biosystems, Carlsbad, Calif.). Also, the same set of 6 index libraries were pooled together in equal concentration to avoid any variation and run in two lanes of Genome analyzer (Illumina, Inc, San Diego, Calif.) to validate the presented metal based DNA fragmentation system. In one embodiment, three different bar-coded genomic DNA libraries (E. coli, Human and Mouse) were prepared using Nextera™ DNA Sample Prep Kit (Illumina®-Compatible) according to manufacturer's instruction (EPICENTRE Biotechnologies, Madison, Wis. 53713). These libraries were also pooled together in equal concentration and were sequenced on Genome analyzer (Illumina, mc, San Diego, Calif.).

### Data Analysis

Mapping. Reads were aligned to reference genomes (Genbank id, hgl9 [ucsc]. mm9 [ucsc]) using Bowtie v0.12.7¹⁵. Only uniquely aligning reads were reported and reads that mapped multiply to the genome were discarded. Up to 2 mismatches were allowed in the alignments.

Random Read Selection and Data Normalization. For each organism, reads were randomly selected from each sequenced sample until the total number of uniquely-mapped reads was equal between fragmentation methods and sequence technologies.

Simulated Random Read Generation. Reads were randomly generated for E. coli to compare coverage and start biases. Fragmentation start sites were chosen using a uniform distribution. A strand direction was also randomly chosen for each unique fragmentation start site. Four million random reads were generated. Simulated reads were mapped with the same methods used in sequenced samples.

Coverage Statistics. Coverage depth and breadth was found using genomeCoverageBed from BEDTools¹⁶. Coverage in GC rich, neutral and poor regions was determined by looking at coverage in 100 bp windows. GC content for each 100 bp window was generated using FastaFromBed, a tool in the BEDTools suite, by dividing the total number of G/C's in each window by the window size (100 bp). Windows were binned into GC rich (GC 60%), (40%<GC<60%) neutral and poor (GC<40%) regions. Average coverage depth and breadth were found in each region by taking the average of all windows within each bin.

Substitution Rates. Substitution rates were determined by comparing each matched base from reads to the corresponding base in the reference sequence. If the read base and the reference base disagreed, it was considered a substitution. The total number of substituted bases was divided by the total number of sequenced bases to yield the total substitution rate.

Regions Flanking Fragmentation Site. A bed file containing elements 20 bp in length that flanked the fragmentation start site was created. FASTA sequences were generated using fastaFromBed, a tool from the BEDTools suite. The frequency of each nucleotide as a function of position in the flanking region was determined using a custom pen script. The GC content of each flanking region was calculated from the FASTA sequence by dividing the total number of G/C and dividing by the length of the sequence.

In some methods, the fragmentation method was optimized and validated for automatable preparation of libraries for SOLiD and Illumina platforms. The influence of temperature, oxygen, time, concentration of reagents, and input DNA on the efficiency of DNA fragmentation was characterized. This metal-based fragmentation is time-dependent, and it allows the isolation of fragments with sizes from 100 bp to 10,000 bp. Also performed was a comparison of library construction by this metal based DNA fragmentation method with conventional DNA fragmentation as well as transposase-mediated DNA fragmentation methods.

### EXAMPLE 2

### DNA Fragmentation Conditions

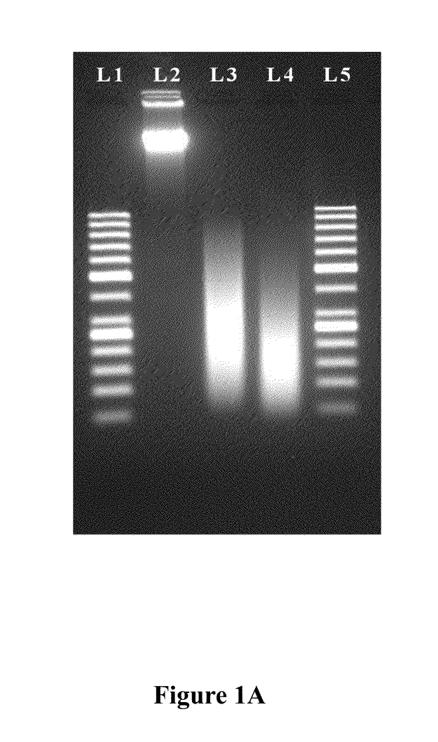

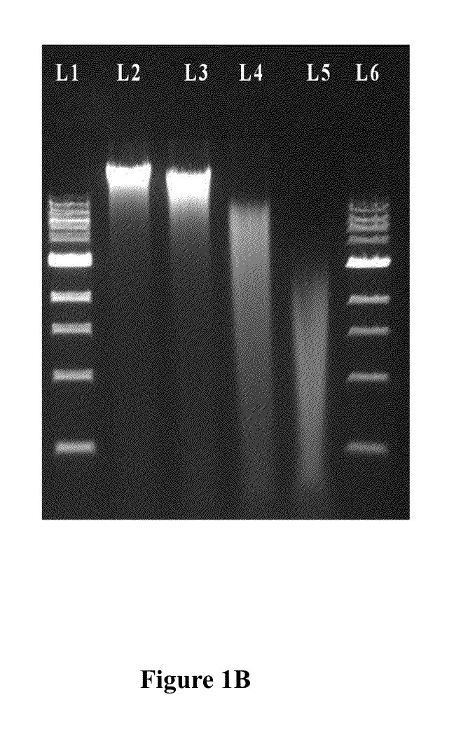

5 ug of DNA was incubated with equimolar concentrations of CuSO₄ and sodium ascorbate (2-10 mM) at room temperature for 30 minutes to examine the DNA fragmentation. Fragmented DNA was purified with charge switch beads, resolved in 2% agarose gel and visualized in SYBR Gold as described above. As shown by agarose gel, when reagent concentration increases, the fragmentation of DNA also increases; in fact, the fragmenting of the DNA can be fine-tuned with reagent concentration from a smear to a band. At 6 mM equimolar concentrations of both reagents, and with an incubation time of 30 minutes, the DNA was fragmented in the range of 100-200 bp, the required range for SOLiD sequencing (FIG. 1A).

DNA was incubated with equimolar concentrations of CuSO₄ and sodium ascorbate at 4 mM to obtain fragments in size range of 200-400 bp, the range required for library preparation on the Illumina platform (FIG. 1A).

In another experiment, using 2 ug DNA and copper and ascorbate at 4 mM concentrations the incubation times (5, 10, 15, 30, 60 minutes) were varied. At 5 and 10 minute incubation times, a smear from 100 bp to 2 kb was observed along with some un-fragmented DNA. Under these conditions, metal-based fragmentation of 2 ug DNA into the size range of 100-400 bp requires at least 20 minutes.

In addition, by varying the reagent concentration and incubation time, the implementation of this metal-based fragmentation for 2 kb, 5 kb and 10 kb size DNA fragments for mate-pair libraries was used so that this method can be utilized for both SOLiD and Illumina platforms. The DNA was incubated at equimolar concentrations of copper and ascorbate at 1.5 mM for 10 minutes to generate an average DNA size range of 2 kb, 0.75 mM for 30 minutes to generate an average DNA size range of 5 kb, and 0.5 mM for 10 minutes to generate an average DNA size range of 10 kb (FIG. 1B). Given the multi-step protocol used for mate-pair library preparation, which involves purification of DNA at every step, mate-pair libraries requires high DNA input (10-20 tg). Therefore, the fragmentation efficiency of the metal-based method for DNA inputs up to 50 ug was evaluated.

The present method can also be used for higher DNA input amounts without changing the concentration of reagents.

It has thus been discovered that a range of transition metal salt (TMS″)/reducing agent (“RA”) concentrations can be used. Using CuSO₄/sodium ascorbate as an exemplary sole reducing agent, it was found that TMS to RA can be present in an equimolar ratio, or in the range of about 0.5 to 1 to 2:1. The size of the fragments generated can be controlled by increasing the concentrations of the TMS/RA, and by controlling the time of incubation, e.g. between 10 and 30 minutes, or between 5 and 60 minutes. Shorter incubation times can be compensated by higher concentrations of TMS/RA. A starting sample with a variety of large DNA molecules, even entire chromosomes, which may be on the order of hundreds of megabases in size, can be used. The inventive methods result in a high yield of fragments (˜40% out of total DNA concentration of fragmented DNA) that fall within a size range useful for library preparation and massively parallel sequencing, e.g. hundreds of base pairs in length, or thousands of base pairs in length. If, for example, a 300 by fragment is desired, ˜40% of the fragmented DNA sample will be in the fragment range of approximately 300 by in length (e.g. +/−10%). However, the amount of cleaved DNA in required fragment size range in comparison to starting input DNA sample is low (10% of total input DNA concentration used for fragmentation) which might be due to degradation of input DNA in to nucleotides. This population of 300 by fragments, i.e. the desired fragment population, can be recovered from the reaction mixture and used for library preparation. Recovery of selected size fragments can be, as demonstrated here, by gel electrophoresis, or other sizing methods such as chromatography can be used. The process generally will be adjusted as described below to produce a population of a certain size of fragments.

The present methods were also used to determine whether Cu²⁺ can be used for DNA fragmentation without reducing it to Cu¹⁺ by using only CuSO₄. Cu²⁺ is not as effective as Cu¹⁺ and it is highly preferred to reduce Cu²⁺ to Cu¹⁺ with an appropriate reducing agent (in this case, ascorbate) for DNA fragmentation. This result was further validated by decreasing the concentration of ascorbate from 4 mM (an equimolar mix with CuSO₄) to 0.1 mM while keeping CuSO₄ concentration constant (4 mM); under these conditions, a significant decrease in DNA fragmentation was observed, suggesting that Cu¹⁺ can fragment DNA more effectively.

The generation of the oxidative species thought to be responsible for fragmentation is catalyzed by transition metals. Thus, Cu²⁺ was replaced with Fe³⁺ to assess the effect of Fe³⁺ on the DNA fragmentation. Fe³⁺ was not as effective as Cu²⁺ in producing the smaller DNA fragments required for next-generation platforms and results were consistent with previous observations in the literature¹⁸⁻¹⁹. In the same vein, the effect of degassing on metal-based DNA fragmentation was assessed, as oxygen radicals generated during the reduction of O₂ can attach to deoxyribose residues to produce damaged bases or strand breaks²⁰. Very low efficiency of fragmentation occurs when DNA samples were fragmented using the metal-based system with continuous degassing, which suggests that fragmentation of DNA may indeed involve reactive oxygen species²⁰.

The effect of different incubation temperatures (25° C., 37° C., 45° C. and 60° C.) on the efficiency of metal-based DNA fragmentation was also evaluated. Temperature variation did not have a significant effect on metal-based DNA fragmentation, and it was preferred to use room temperature to make this protocol simple and easy to use.

### EXAMPLE 3

### Deep Sequencing of Libraries Prepared from Metal-Based DNA Fragmentation

The applicability of the metal-based DNA fragmentation system was validated by comparing the sequencing data of libraries prepared by this method to libraries prepared from fragments created using the Covaris Adaptive Focused Acoustics (AFA)™ technology (a widely used method of DNA shearing) for three different genomes (E. coli, Human and Mouse). In Illumina sequencing, 1.87 million reads from AFA-prepared E. coli library and 2.12 million reads from the E. coli library that employed metal-based fragmentation were mapped to the E. coli genome; 95.29% and 94.95% of the E. coli genome was covered respectively, with a mean coverage depth of 9.52× (Table 1).

TABLE 1
Summary of read alignments, overall coverage and substitution rates for
three genomes (E. coli [1-5] Human [6-9] and Mouse [10-13]) from two
next generation platforms (IlluminaGenome Analyzer & SOLiD).

| | | Frag- | | | |
|---|---|---|---|---|---|
| | | mentation | Total | Unique | % Unique |
| Sample | Sequencer | Method | Mapped | Starts | Starts |
| 1. | Illumina | Covaris | 1869309 | 1577063 | 84.37% |
| 2. | Illumina | Copper | 2122423 | 1684169 | 79.35% |
| 3. | Solid | Covaris | 11172013 | 5446901 | 48.75% |
| 4. | Solid | Copper | 1166869 | 881885 | 75.58% |
| 5. | N/A | Simulated | 3881698 | 3151856 | 81.20% |
| | | Random | | | |
| 6. | Illumina | Covaris | 1302613 | 1256936 | 96.49% |
| 7. | Illumina | Copper | 2293382 | 2213090 | 96.50% |
| 8. | Solid | Covaris | 10921280 | 10421365 | 95.42% |
| 9. | Solid | Copper | 863705 | 721151 | 83.50% |
| 10. | Illumina | Covaris | 1074401 | 1011479 | 94.14% |
| 11. | Illumina | Copper | 2807589 | 2741243 | 97.64% |
| 12. | Solid | Covaris | 9306326 | 8825536 | 94.83% |
| 13. | Solid | Copper | 2153617 | 1736992 | 80.65% |
| Sample | % Unique | Reads | Percent | Coverage | Substitution |
| (Cont.) | Starts | Analyzed | Coverage | Depth | Rate |
| 1. | 84.37% | 881885 | 95.29% | 9.52 | 0.43% |
| 2. | 79.35% | 881885 | 94.95% | 9.52 | 0.62% |
| 3. | 48.75% | 881885 | 96.01% | 9.14 | 0.06% |
| 4. | 75.58% | 881885 | 93.48% | 9.14 | 0.18% |
| 5. | 81.20% | 881885 | 97.37% | 9.52 | N/A |
| 6. | 96.49% | 721151 | 1.13% | 0.01 | 0.46% |
| 7. | 96.50% | 721151 | 1.14% | 0.01 | 0.66% |
| 8. | 95.42% | 721151 | 1.04% | 0.01 | 0.08% |
| 9. | 83.50% | 721151 | 1.09% | 0.01 | 0.27% |
| 10. | 94.14% | 1011479 | 1.08% | 0.01 | 0.57% |
| 11. | 97.64% | 1011479 | 1.80% | 0.02 | 0.75% |
| 12. | 94.83% | 1011479 | 1.82% | 0.02 | 0.12% |
| 13. | 80.65% | 1011479 | 1.60% | 0.02 | 0.21% |

Similarly in SOLiD sequencing, 1.11 million reads from AFA-prepared libraries and 1.16 million reads from metal-based fragmentation libraries were mapped and 96.01% and 93.48% of the E. coli genome was covered respectively, with a mean coverage depth of 9.14× (Table 1; FIG. 1A-B). The coverage depth is almost identical for E. coli genome with libraries prepared by the metal-based method and more conventional AFA-based DNA shearing method. FIG. 2 illustrates the comparison of coverage depth (50 kb coverage window) for the E. coli genome from sequencing data of both Illumina (FIG. 2A) and SOLiD (FIG. 2B) platforms for libraries prepared from products of the two different fragmentation methods. Human and mouse genomes were covered 1-2% for both kind of libraries (Table 1). Although the number of reads is insufficient to assess the coverage depth for such a complex genomes, both genomes showed a good correlation in coverage depth in this 50 kb coverage window for both libraries (Table 1).

Further, unique start sites were investigated to assess any sequence-specific biases in DNA fragmentation. The percentage of unique start sites was slightly higher for libraries prepared with products of the AFA technology in comparison to those made using metal-based fragments in Illumina data (84% unique starts for AFA, 79% for metal-based) for the E. coli genome. However, the unique start percentage was lower for libraries prepared using AFA-generated fragments (48%) in comparison to libraries prepared with metal-based fragmentation (75%) in SOLiD data, an artifact of deep sequencing a small genome (Table 1).

Additionally, base-specific biases in the start of read were assessed by calculating the percentage of each base at the read start site. Results were measured and compared between (1) Illumina with Covartis™ AFA acoustic shockwave fragmentation, (2) Illumina with metal-based (copper) fragmentation, (3) SOLiD sequencing with AFA fragmentation, (4) SOLid sequencing with metal-based fragmentation, (5) Illumina sequencing samples prepared by Nextera™ sample preparation kit, and (6) randomly generated fragmentation (calculated). These 6 measurements were taken for three genomes (E. coli, mouse, and human).

In general, results comparing 6 techniques in three genomes showed equivalence between the present copper method and acoustic shockwave shearing. In each case, the percentage of adenine bases is higher, and the percentage of thymine bases is lower, at the first position of the reads in both kinds of libraries (Fragmentation by COVARIS and metal based) for all three different genomes in data generated by Illumina sequencing platform. The percentage of adenine is slightly higher at the first base in libraries prepared from metal based fragmentation in comparison to libraries built from AFA-generated fragments for both human and mouse genomes. However, this first base bias in the data generated by SOLiD which uses blunt-end adaptor ligation protocol for library preparation instead of dA-tailing was not observed. Thus, the starting base preference does not appear to be a consequence of the fragmentation methodology, but most likely due to the preferential dA-tailing or the ligation step of library preparation²¹. In case of data generated using Nextera library preparation protocol, it was observed that base-specific bias occurs in the start of read up to first 15 bases of the read in all three different genomes. The pattern of bases specificity is resembling to the reported consensus insertion site, AGNTYWRANCT (SEQ ID NO: 1), of the native Tn5 transposase^{7,22}. These sequence specific biases looks more prominent in mouse and human genomes in comparison to E. coli genome which is merely due to slight higher percent of AT than GC bases in mouse and human genome than AT/GC balanced genome of E. coli, in general, shown by random reads generated from three different genomes.

GC-biases during fragmentation were evaluated by considering the GC content of the 20 bp flanking the start site. Similar average coverage depth across the genome and coverage in AT-rich and GC-rich regions for all three genomes was observed (Table 2).

TABLE 2
GC-dependent coverage statistics for three genomes (E. coli,
Human and Mouse) from two next generation platforms (Illumina
Genome Analyzer & SOLiD).

| | Average | | | | | |
|---|---|---|---|---|---|---|
| | Depth of Coverage | | | Coverage Across Genome | | |
| | | 40% < | | | 40% < | |
| | GC < | GC < | GC > | GC < | GC < | GC > |
| | 40% | 60% | 60% | 40% | 60% | 60% |
| E. coli Illumina | 3.25 | 9.93 | 12.17 | 80.11% | 96.95% | 94.16% |
| Covaris | | | | | | |
| E. coli Illumina | 3.19 | 9.84 | 13.22 | 79.31% | 96.60% | 94.45% |
| Copper | | | | | | |
| E. coli Solid | 6.33 | 9.45 | 8.95 | 86.27% | 97.18% | 94.32% |
| Covaris | | | | | | |
| E. coli Solid | 4.32 | 9.53 | 10.34 | 71.25% | 95.70% | 94.23% |
| Copper | | | | | | |
| E. coli Simulated | 9.62 | 9.55 | 9.15 | 98.09% | 97.68% | 93.76% |
| Random | | | | | | |

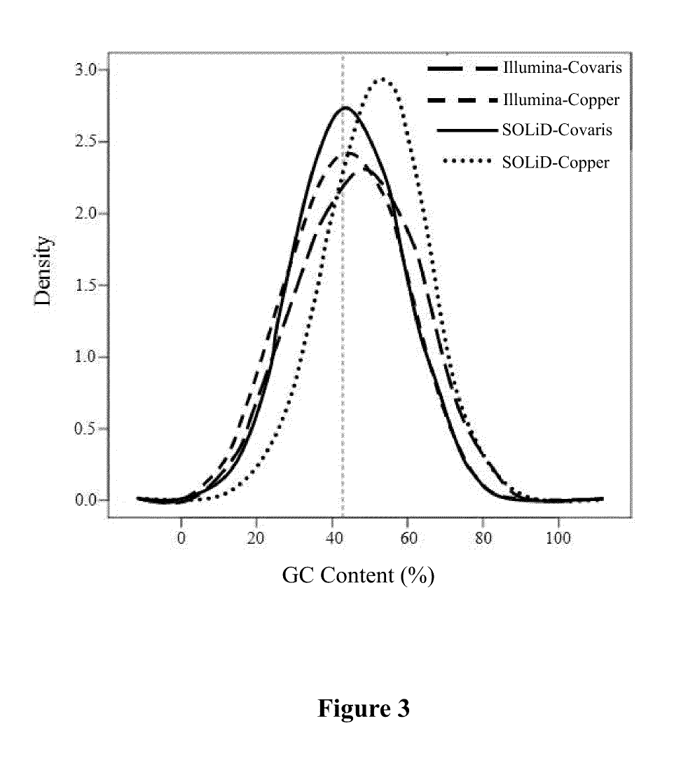

FIG. 3 illustrates a slight shift of read density in regions of higher GC content for SOLiD data in the human all three genomes (E. coli, mouse, human); this phenomenon is more prominent in human and mouse samples. For all three genomes, SOLiD sequencing data is slightly biased away from neutral GC content to higher GC content. E. coli samples showed a general under-representation in GC-poor regions in all sequence platforms and fragmentation methods. Mouse samples showed a preference for GC neutral regions in SOLiD datasets. Illumina data are similar for both fragmentation methods, with a slightly higher representation of GC rich regions in AFA-generated libraries.

Because pyrimidine bases are more sensitive to oxidative DNA damage than are purines²³ the purine (AG) content of the 20 bp region flanking the read start site was computed to study the effect of copper on pyrimidine damage in these genomes. The purine content at the read start site was similar in both kinds of libraries for the three different genomes. The error rate for reads obtained from all six genomic libraries (three species, prepared with AFA-generated fragments versus those prepared from metal-based fragmentation) was also calculated and no significant differences were found, which suggests minimal base damage of DNA during metal-based fragmentation under the optimized conditions used here (Table 1).

### EXAMPLE 4

### Fragmentation of 0.5 ug E. coli Genomic DNA

0.5 ug of E-coli Genomic DNA was incubated with equimolar concentrations of CuSO₄ and sodium ascorbate (1.4 mM) at room temperature for 5 minutes to examine the DNA fragmentation. The reaction was stopped by adding 50 mM EDTA, heated at 37 C for 5 min., and then fragmented DNA was purified with carboxyl terminated beads and resolved in an Agilent area under the curve Bioanalyzer high sensitivity chip as described above. As shown by bioanalyzer trace, (FIG. 4), the resultant E. coli DNA fragments range in size from 100 nt to ˜12,000 nt. The Table below shows fragment size ranges (from A to B) and the corresponding minimum yield (Y>) for the amount of the fragment present in the treated sample. For example, row 1 shows that for a size range of 100-200 nt a yield greater than 4% was obtained. 15% of fragments measured between 100 nt and greater than 9500 nt in length. In this case, minimum fragment size analyzed was 100 nt, the average input (sample) polynucleotide length was >9,500, and the yield of all fragments was 15%. The fragment size most predominant in the treated sample in this example, as indicated by the Table below, was 100-200 nt in length.

| FROM A | TO B | Y> |
|---|---|---|
| 100 | 200 | 4.0% |
| 250 | 300 | 2.0% |
| 600 | 800 | 1.0% |
| 2,000 | 4,000 | 0.6% |
| 8,000 | 9,500 | 0.1% |

### EXAMPLE 5

### Fragmentation of 1.0 ug E. coli Genomic DNA

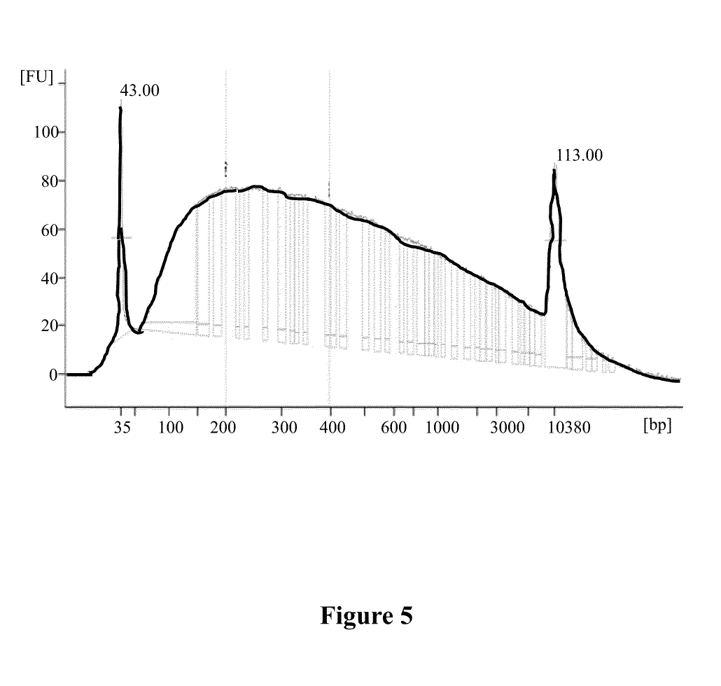

1.0 ug of E-coli Genomic DNA was incubated with 1.4 mM CuSO4 and 1.7 mM sodium ascorbate at room temperature for 5 minutes to examine the DNA fragmentation. The reaction was stopped by adding 50 mM EDTA, heated at 37 C for 5 min and then fragmented DNA was purified with carboxyl terminated beads and resolved in the bioanalyzer high sensitivity chip described above. As shown by bioanalyzer trace, (FIG. 5). The fragmented E-Coli DNA ranges from 100 nt to ˜12,000 nt.

These variables and those in the table below may be interpreted as in Example 4.

| FROM A | TO B | Y> |
|---|---|---|
| 100 | 200 | 4.0% |
| 250 | 300 | 2.0% |
| 600 | 800 | 1.0% |
| 2,000 | 4,000 | 0.6% |
| 8,000 | 9,500 | 0.1% |

### EXAMPLE 6

### Fragmentation of 3.0 ug E. coli Genomic DNA

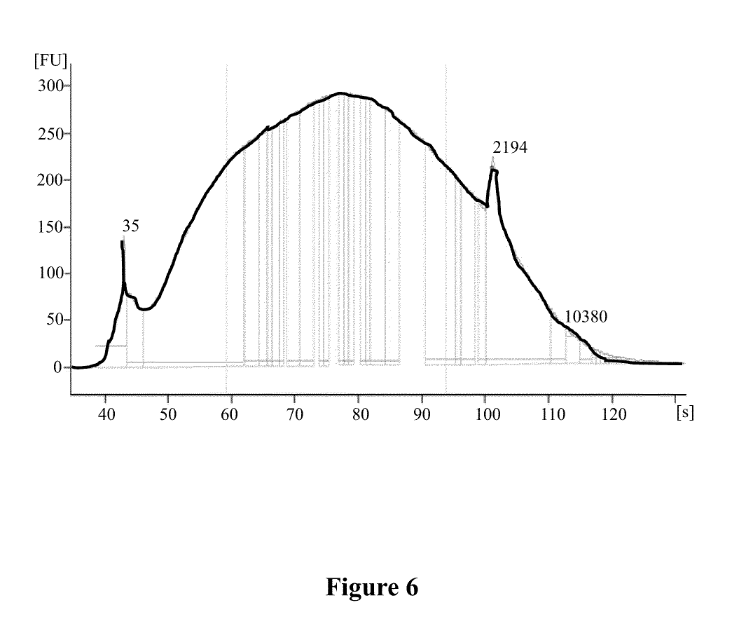

Genome sequencing centers or laboratories are currently obligated to use two different types of instrument to shear the DNA for fragment libraries and for mate-pair libraries (e.g., a Covaris AFA instrument for fragment libraries, and a Digilab Hydroshear for mate-pair libraries). There is no single method or instrument available that can effectively perform both tasks 3.0 ug of E-coli Genomic DNA was incubated with 1.4 mM CuSO₄ and 2.0 mM of sodium ascorbate at room temperature for 5 minutes to examine the DNA fragmentation. The reaction was stopped by adding 50 mM EDTA, heated at 37 C for 5 min and then fragmented DNA was purified with carboxyl terminated beads, resolved in the Agilent bioanalyzer high sensitivity chip described above. As shown by bioanalyzer trace, (FIG. 6). The fragmented E-Coli DNA ranges from 100 nt to ˜12,000 nt.

| FROM A | TO B | Y> |
|---|---|---|
| 100 | 200 | 0.5% |
| 250 | 300 | 0.25% |
| 600 | 800 | 0.2% |
| 2,000 | 4,000 | 0.11% |
| 8,000 | 9,500 | 0.025% |

These variables and those in the table below may be interpreted as in Example 4.

### EXAMPLE 7

### Fragmentation of 0.5 ug Human Genomic DNA

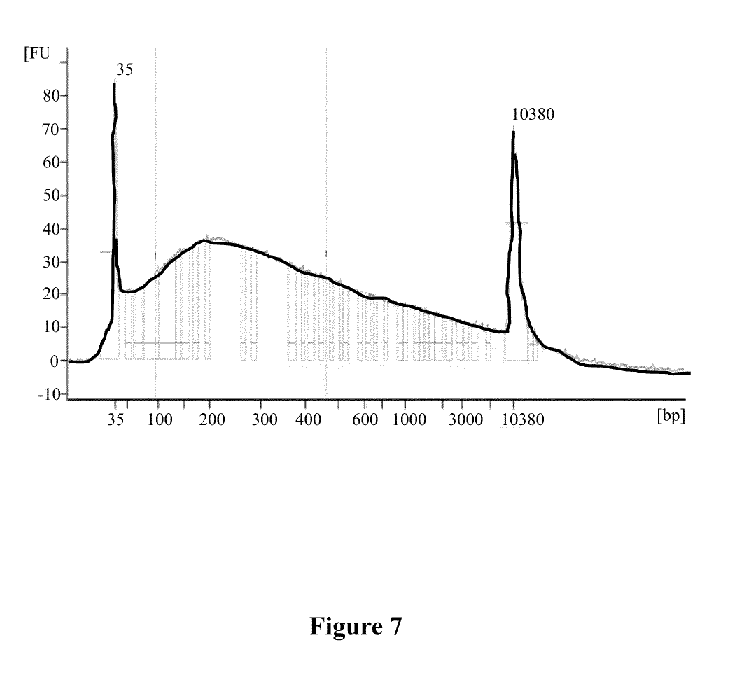

0.5 ug of Human genomic DNA was incubated with 1.4 mM CuSO₄ and 2.0 mM of sodium ascorbate at room temperature for 5 minutes to examine the DNA fragmentation. The reaction was stopped by adding 50 mM EDTA, heated at 37 C for 5 min and then fragmented DNA was purified with carboxyl terminated beads, resolved in the bioanalyzer high sensitivity chip described above. As shown by bioanalyzer trace, (FIG. 7). The fragmented DNA ranges from 100 nt to ˜12,000 nt.

| FROM A | TO B | Y> |
|---|---|---|
| 100 | 200 | 7.0% |
| 250 | 300 | 2.5% |
| 600 | 800 | 1% |
| 2,000 | 4,000 | 0.5% |
| 8,000 | 9,500 | 0.1% |

These variables and those in the table below may be interpreted as in Example 4.

### EXAMPLE 8

### Fragmentation of 1.0 ug Human Genomic DNA

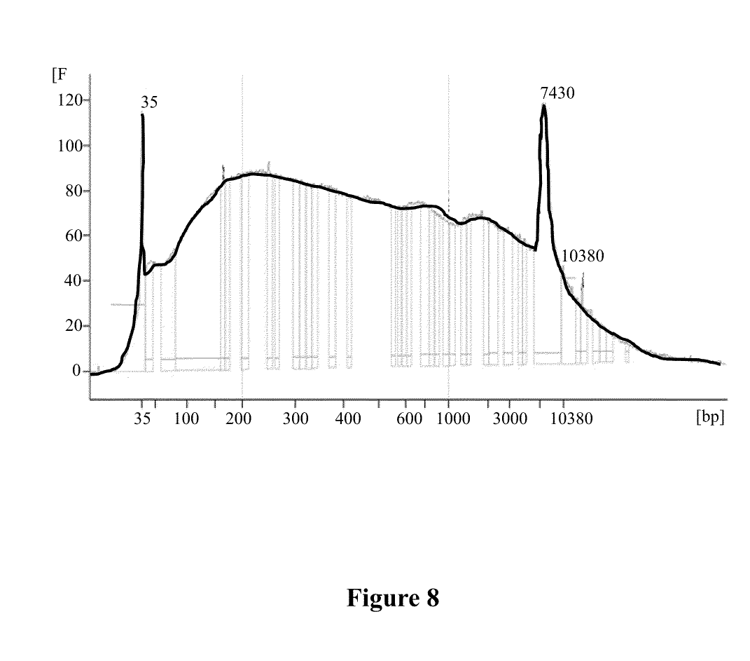

1.0 ug of Human Genomic DNA was incubated with 1.4 mM CuSO4 and 2.5 mM of sodium ascorbate at room temperature for 5 minutes to examine the DNA fragmentation. The reaction was stopped by adding 50 mM EDTA, heated at 37 C for 5 min and then fragmented DNA was purified with carboxyl terminated beads, resolved in the bioanalyzer high sensitivity chip described above. As shown by bioanalyzer trace, (FIG. 8). The fragmented DNA ranges from 100 nt to ˜12,000 nt.

| FROM A | TO B | Y> |
|---|---|---|
| 100 | 200 | 3.0% |
| 250 | 300 | 1.2% |
| 600 | 800 | 0.6% |
| 2,000 | 4,000 | 0.4% |
| 8,000 | 9,500 | 0.08% |

These variables and those in the table below may be interpreted as in Example 4.

### EXAMPLE 9

### Fragmentation of 3.0 ug Human Genomic DNA

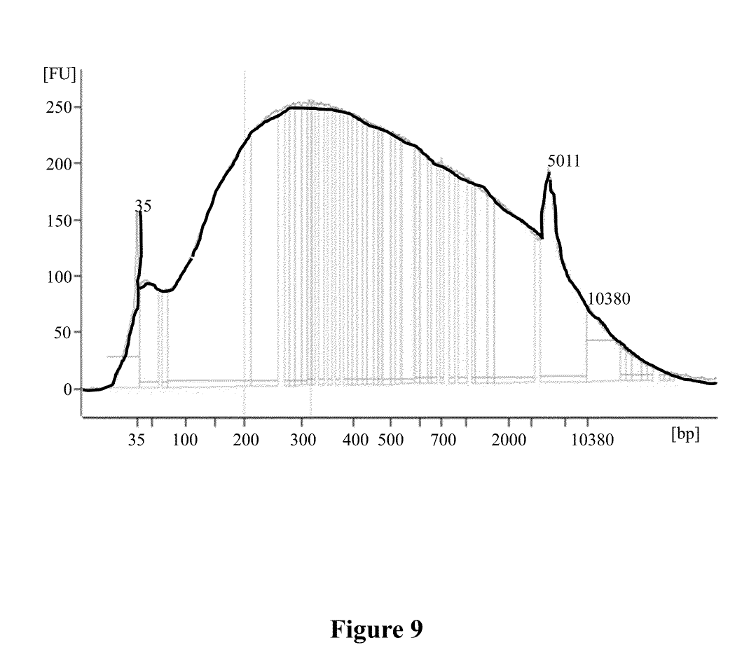

3.0 ug of Human Genomic DNA was incubated with 1.4 mM CuSO₄ and 2.8 mM of sodium ascorbate at room temperature for 5 minutes to examine the DNA fragmentation. The reaction was stopped by adding 50 mM EDTA, heated at 37 C for 5 min and then fragmented DNA was purified with carboxyl terminated beads, resolved in the Agilent Bioanalyzer high sensitivity chip described above, as shown by bioanalyzer trace, (FIG. 9). The fragmented DNA ranges from 100 nt to ˜12,000 nt.

| FROM A | TO B | Y> |
|---|---|---|
| 100 | 200 | 0.8% |
| 250 | 300 | 0.5% |
| 600 | 800 | 0.23% |
| 2,000 | 4,000 | 0.13% |
| 8,000 | 9,500 | 0.03% |

These variables and those in the table below may be interpreted as in Example 4.

### EXAMPLE 10

### Automation Using Robotics

In this example, an automated liquid handling robot is used to prepare libraries for sequencing. The input material is size-selected DNA isolated from the solution based fragmentation technology described above. Sequence ready samples were made from the solution and isolation steps. As described above, these fragmentation techniques eliminate the troublesome, one sample at a time processing performed by sonication.

The present methods comprise a combination of fragmentation, DNA size selection of desired size using paramagnetic carboxylic-acid coated beads, which are commercially available, and automated DNA library preparation. The library prepared is suitable for use on any next generation sequencing platform such as Illumina's HiSeq, Roche/454's GS FLX Titanium, Life Technologies's SOLiD, Pacbio and/or any other platforms that needs library preparation.

Library preparation for each next-generation sequencing platform has similar protocols. The steps involved in DNA library preparation for a next-generation sequencing platform starts from (1) random fragmentation or shearing of genomic DNA, (2) the selection of desired size range for specific platform, (3) end polishing and adaptor ligation, (4) enrichment, (5) purification.

The present method may be carried out in the following steps:

1. Obtain a blood or tissue sample;
2. Add a mixture of Cu2+ ions and ascorbic acid;
3. Incubate the mixture for time sufficient to generate appropriate length fragments. The incubation time is directly proportional to the ionic concentration of mixture in step 2, directly proportional to the length of the length of the starting polynucleic acids, and inversely proportional to the resulting average fragment length.
4. Stop the reaction with a chelator such as EDTA;
5. Add beads in solution to adsorb DNA fragments;
6. Remove the beads with bound DNA;
7. Remove DNA fragments from the beads.

Each of the above steps can be pre-programmed into an automated device. The incubation step 3 can be as short as about 5 minutes or less, with increased concentrations of the mixture in step 2. The reaction may also be carried out at elevated temperatures. The chelator in step 4 will bind the Cu ions and prevent further fragmentation. The beads can be removed from the solution in step 6 by a filtration, pipette, or magnetic process. Beads can be used which bind DNA under certain buffer conditions, then release the DNA under altered buffer conditions, such as by addition of salt to the bead mixture.

The following is an exemplary protocol:

### Shearing

A copper/ascorbate shearing solution, comprising 5 mM CuSO₄ and 50 mM NaAsc is prepared. 20 uL 50 mM NaAsc is added to 100 ul of 5 mM CuSO4 and mixed well until the solution is bright yellow. 16 uL of this copper/ascorbate solution is mixed with 34 uL DNA to make 50 uL total reaction mixture. This is incubated for 5 mins. Then, 5 uL of 0.5M EDTA is added and mixed. This is incubated for 5 mins at 37° C.

### Purification and Shearing

15 uL of beads are added to the sample from the preceding step and mixed thoroughly. To this is added 150 uL of BindALL Buffer, (Nordiag ASA, Oslo, Norway) band the sample was incubated. The magnetic beads (carboxylic acid magnetic beads from Nordiag) are pelleted with a magnet and supernatant is removed. The beads are washed and resuspended in Release Buffer 1 (provided by Nordiag ASA) (50 μL). This is mixed, incubated, pelleted, washed and resuspended with Release Buffer 2 (50 μL). Beads are pelleted with a magnet and the supernatant saved. 15 uL of new beads are added to 50 uL of Release Buffer 2 supernatant. 150 uL of BindAll Buffer is added to the Release Buffer 2 supernatant. Beads are pelleted with magnet and supernatant removed. The pellet is washed and supernatatant removed. DNA is eluted in 45 uL of water. (Size Selected Sample)

### Library Preparation

The library preparation steps followed standard procedures of end repair, dA-tailing, and adapter ligation. Adaptor purification is done by adding 15 uL of beads to the sample and mixing thoroughly. DNA is bound to magnetic beads using the manufacturer supplied buffer (BindALL Buffer), which is added to the sample, and the beads are pelleted, washed and resuspended. The adapter ligated sample is amplified by PCR, using the following components.

| | | |
|---|---|---|
| Adaptor Ligated Sample | 19 | uL |
| Primer 1.0 | 2 | uL |
| Primer 2.0 | 2 | uL |
| Index # | 2 | uL |
| Phusion High Fidelity PCR Master Mix | 25 | uL |
| Sterile H₂0 for final Vol | 0 | |
| Total Volume | 50 | uL |

| CYCLE STEP | TEMP | TIME | CYCLES |
|---|---|---|---|
| Initial Denaturation | 98° C. | 30 sec | 1 |
| Denaturation | 98° C. | 10 sec | 14 |
| Annealing | 65° C. | 30 sec | 14 |
| Extension | 72° C. | 30 sec | 14 |
| Final Extension | 72° C. | 5 min | 1 |
| | 4° C. | Hold | 1 |

### PCR Cycling Conditions

Post PCR size selection is continued if amplification is present. This is also done using by adding beads to the sample, followed by binding buffer, pelleting the beads and suspending them in release buffer. Then they are pelleted again, using a magnet, and supernatant is removed. The beads are resuspended in release buffer, mixed and pelleted and resuspended in a post PCR release buffer (Nordiag ASA, Oslo, Norway), which releases the amplified products from the beads.

### REFERENCES

1. J. Shendure and H, Ji, Nat Biotechnol 26(10), 1135 (2008).
2. M. L. Metzker, Nat Rev Genet. 11(1), 31 (2010).
3. E. Pettersson. J. Lundeherg. and A. Ahmadian, Genomics 93 (2), 105 (2009).
4. E. Farias-Hesson, J. Erikson, A. Atkins et al., J Bioimed Biotechnol 2010, 617469 (2010).
5. Y. R. Thorstenson, S. P. Hunicke-Smith, P. J. Oefner et al., Genome Res 8 (8), 848 (1998).
6. Joseph Sambrook and David W. Russell, Cold Spring Harb Protoc 2006 (4), pdb.prot4539 (2006).
7. Andrew Adey, Hilary Morrison, Asan et al., Genome Biology 11(12), R119 (2010).
8. T D Tullius and BA Dombroski Science 230 (4726) 679 (1985)
9. Shyh-Horng CHIOU, Journal of Biochemistry 94 (4), 1259 (1983).
10. Matt S Melvin, John T Tomhnson, Gilda R Saluta et al. Journal of the American Chemical Society 122 (26), 6333 (2000).
11. Bhavani Balasubramanian. Wendy K. Pogozeiski, and Thomas D. Tullius. Proceedings of the National Academy of Sciences of the United States of America 95 (17), 9738 (1998).
12. T D Tullius and B A Dombroski, Proceedings of the National Academy of Sciences of the United States of America 83 (15), 5469 (1986).
13. M. A. Price and T. D. Tullius, Methods in Enzymology 212, 194 (1992).
14. J A Latham and T R Cech, Science 245 (4915), 276 (1989).
15. B. Langmead, C. Trapnell, M. Pop et al., Genome Biol 10 (3), R25 (2009).
16. Aaron R. Quinlan and Ira M. Hall, Bioinformatics 26 (6), 841 (2010).
17. J. L. Sagnpant and K H Kraemer, J Biol Chem 264 (3) 1729 (1989)
18. C. J. Reed and K. T. Douglas, Biochem Biophs Res Comma? 162 (3), 1111 (1989).
19. P. Tachon, Free Radic Res Commun 7 (1), 1 (1989).
20. Lawrence J. Marnett, Carcinogenesis 21(3), 361 (2000).
21. Iwanka Kozarewa, Zemin Ning, Michael A. Quail et al., Nat Meth 6 (4), 291 (2009).
22. I. Y. Goryshin, J. A. Miller, Y. V. Ku et al., Prot Natl Acad Sci USA. 1998 Sep. 1; 95(18):10716-21 95 (18), 10716 (1998).
23. R. Teoule and J. Cadet, Mol Biol Biochem Biophys 27. 171 (1978).
24. Sergey A. Kazakov, Tatiana G. Astashkina, Sergey V. Mamaev et al., Nature 335 (6186), 186 (1988).

### CONCLUSION

The above specific description is meant to exemplify and illustrate the method and should not be seen as limiting the scope of the invention, which is defined by the literal and equivalent scope of the appended claims. Any patents or publications mentioned in this specification are indicative of levels of those skilled in the art to which the patent or publication pertains as of its date and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material to which is referred.

What is claimed is:

1. A method for generating polynucleic acid fragments having a first sized fragment population and one or more second sized fragment populations, from a starting polynucleic acid sample wherein sample polynucleic acids have an average length of at least 1200 nt, comprising the steps of:
(a) contacting the polynucleic acid sample with a reducing agent and a transition metal in a solvent to form a mixture; and,
(b) incubating the mixture from step (a) for a predetermined time to cause random fragmentation of sample polynucleic acids substantially along their length,
i. wherein the yield of fragments is at least 0.2% of the sample, and
ii. wherein the amount fragments in the first sized fragment population is greater than an amount of fragments in a second sized fragment populations.

2. The method of claim 1, wherein the transition metal is an ion.

3. The method of claim 2, wherein the ion is selected from the group consisting of a copper ion and an iron ion.

4. The method of claim 3, wherein the transition metal ion is a cupric ion.

5. The method of claim 1, wherein the reducing agent is an ascorbic acid reducing agent.

6. The method of claim 5, wherein the reducing agent is sodium ascorbate.

7. The method of claim 1, wherein the solvent is an unbuffered aqueous solution.

8. The method of claim 7, wherein the solution is in water.

9. The method of claim 1, wherein the method does not include contacting the mixture with piperidine.

10. The method of claim 1, further comprising isolating from the mixture the first sized fragment population.

11. The method of claim 1, wherein said first population comprises fragments in a size range from about 100-200 nt, about 200-300 nt, about 250-300 nt, about 250-350, about 200-400 nt, about 300-400 nt, about 600-800 nt, about 2,000-4,000 nt, or 8,000-10,000 nt.

12. The method of claim 1, wherein a second population comprises fragments in a size range from about 100-200 nt, about 200-300 nt, about 250-300 nt, about 250-350 nt, about 200-400 nt, about 300-400 nt, about 600-800 nt, about 2,000-4,000 nt, or 8,000-10,000 nt.

13. The method of claim 1, wherein the first population is defined by a nominal size that varies by less than about 50 nt.

14. The method of claim 1, wherein the yield of fragments comprises a percentage of the total population that is one of 2, 3, 4, 10, 20, 30, 40, 50, 60, 60, 70, or 80.

15. The method of claim 1, wherein the percentage yield of fragments in the first size fragment population is one of 0.5, 1, 2, 5, 10, 15, 20, 25, 30, 35, 40, 45 or 50.

16. The method of claim 1, further comprising the steps of
contacting fragments in the first sized fragment population with a solid support, and allowing said fragments to bind to the solid support;
removing unbound polynucleic acid fragments; then
releasing the bound polynucleic acid fragments.

17. The method of claim 1, further comprising the steps of
attaching fragments in the first sized fragment population to a solid support; and
removing unbound polynucleic acid fragments;

18. The method of claim 17, further comprising the step of end polishing the released polynucleic acid fragments.

19. The method of claim 17, wherein said solid support comprises a bead.

20. The method of claim 17, wherein the steps are automated.

21. A method for generating a population of polynucleic acid fragments in a sample, comprising the steps of:
(a) contacting polynucleic acids in the sample with a reducing agent and a transition metal in an unbuffered aqueous solution to form a mixture; and,
(b) incubating the mixture under conditions that fragment the polynucleic acids into a distribution of fragment sizes.

22. The method of claim 21, wherein the unbuffered aqueous solution is water.

23. The method of claim 22, wherein the transition metal is an ion.

24. The method of claim 23, wherein the ion is selected from the group consisting of a copper ion and an iron ion.

25. The method of claim 24, wherein the transition metal ion is a cupric ion.

26. The method of claim 21, wherein the reducing agent is an ascorbic acid salt or derivative.

27. The method of claim 26, wherein the reducing agent is sodium ascorbate.

28. The method of claim 21, wherein the method does not include a piperidine cleavage step.

29. The method of claim 21, further comprising isolating a subpopulation of polynucleic acids from the distribution of fragment sizes.

30. A method for generating a population of polynucleic acid fragments in a sample, comprising the steps of:
(a) contacting polynucleic acids in the sample, wherein the polynucleic acids have an average length of at least 1200 nt, with a reducing agent and a transition metal in a solvent to form a mixture; and
(b) incubating the mixture under conditions that fragment the polynucleic acids into a distribution of fragment sizes
wherein the method does not include contacting the mixture with piperidine.

31. A composition comprising a polynucleic acid, a transition metal and reducing agent in an unbuffered aqueous solution.

32. The composition of claim 31, wherein said polynucleic acid is genomic DNA, said transition metal is in the form of a transition metal salt and said reducing agent is an ascorbate reducing agent.

33. The composition of claim 32, wherein said transition metal salt and said reducing agent are present in approximately a 1:1 molar ratio.

34. A kit for generating a population of polynucleic acid fragments in a sample comprising (a) a transition metal and reducing agent in an unbuffered aqueous solution and (b) instructions for use.
